(19) United States
(12) Patent Application Publication
(10) Pub. No.: US 2019/0167115 A1
(43) Pub. Date: Jun. 6, 2019

(54) METHOD AND SYSTEM FOR PROCESSING AN IMAGE OF THE TEETH AND GUMS

(71) Applicant: ONARIA TECHNOLOGIES LTD., London (GB)

(72) Inventors: Rojina DORODVAND, London (GB); Hawaa BUDRAA, London (GB); Basaru Rilwan REMILEKUN, London (GB)

(21) Appl. No.: 16/324,176

(22) PCT Filed: Aug. 9, 2017
(86) PCT No.: PCT/EP2017/070246

§ 371 Date: Feb. 8, 2019

(30) Foreign Application Priority Data
Aug. 9, 2016 (EP) 16183461.9

(51) Int. Cl. A61B 5/00

(52) U.S. Cl. CPC A61B 5/0088; A61B 5/4547; A61B 5/682; A61B 5/4842; A61B 5/7275; A61B 5/4552

(57) ABSTRACT

A system for processing an image of the mouth, includes an image capture device; a mouthpiece attachment, configured to attach to the image capture device and mount the image capture device in a fixed position relative to a user's mouth; a display; and a processing unit; wherein the processing unit is configured to: receive mouth image data from the image capture device; identify an area of analysis within the image data corresponding to the teeth and gums; extract a set of image property features from the area of analysis of the image data; pass the image property features through a condition classifier, the condition classifier configured to compare the values of the extracted image property features against pre-set parameters to identify sub-areas of the area of analysis which are indicative of oral health conditions; and send result data corresponding to the identified sub-areas to the display.

### TECHNICAL FIELD

The present invention relates to a system and method for receiving and analysing images of human teeth and gums.

### BACKGROUND

The oral cavity in humans is an indicator of a number of diseases, including periodontal disease, gingivitis, oral cancer and dental cavities/caries. Such conditions affect a large proportion of the population. For example, worldwide, 60-90% of school children and nearly 100% of adults have dental cavities, often leading to pain and discomfort.

The teeth are the hardest substances in the human body. Besides being essential for chewing, the teeth play an important role in speech. Gums are made of a soft skin designed to cover the bones of your teeth and form a tight seal to support the bones and provide a barrier to bacteria. The progression of oral diseases, if left untreated, can have implications for these essential functions of the teeth and gums. For this reason, it is important to regularly monitor the health of teeth and gums to prevent the formation of plaque on areas such as the tooth surfaces which can lead to a range of diseases from oral to systemic.

The monitoring of the condition of teeth and gums can provide benefits in the early identification and treatment of a number of conditions.

Malocclusion is when teeth are not aligning properly. Changes in your mouth can be due to problems such as tumours of the mouth or jaw and have a dramatic effect on the alignment of your teeth hence it is important to monitor the change in the morphology of the alignment of teeth over time.

Tooth sensitivity is when the dentin of the tooth is exposed, inflicting problems such as significant pain and discomfort when encountering certain temperatures or substances. Tooth sensitivity may also be linked to more serious dental problems. Monitoring the morphology, colour, texture of the enamel and gums may be used to track enamel erosion and gum recession over time.

If plaque is allowed to build up, it can lead to further problems, such as dental caries (holes in the teeth), gum disease or dental abscesses, which are collections of pus at the end of the teeth or in the gums. Therefore, monitoring of change in the morphology, colour and texture of plaque on, around and in-between teeth surfaces instantaneously and over time is crucial in identifying the onset of such conditions.

Gingivitis (inflamed and irritated gums), receding gums and periodontal disease may be identified by monitoring of the morphology, colour and texture of the gums over time, with a view to tracking the rate of inflammation.

Monitoring the change in the morphology, colour and texture of the teeth surfaces over time may also be used to track the effectiveness of beauty products and treatments applied to the teeth, such as whitening gel and special toothpastes.

In order for a patient to be able to monitor the condition of their teeth and gums, monitoring systems should preferably allow patients to reliably identify changes in the appearance of the teeth and gums which could be indicative of conditions such as those described above. Preferably such methods should be affordable, non-time consuming, portable, easy to use, require no medical training or expertise and accessible whilst facilitating the reliable and objective identification of changes in oral health.

There are a number of known methods and systems for monitoring the oral health of a patient. These particularly related to methods of identifying levels of plaque on the teeth and gums.

Firstly considering methods of monitoring plaque levels on teeth, disclosing tablets may be used to stain plaque and provide a visual aid for patients to assess their oral health. Although disclosing tablets are relatively accessible and affordable they are time consuming and uncomfortable to use. Furthermore, to go beyond a subjective evaluation of oral health, an indexing system is required to convert the observed plaque coverage to a quantified measure of tooth and gum health. There are several known indices currently used for the estimation of plaque quantities based on coverage area and thickness of observed plaque, including Schick and Ash, Quigley and Hein, Silnes and Loe and the DPMI system. However the use of these indices is time consuming and generally requires an experienced professional to reliably determine the scores.

Regarding methods of monitoring gum health, this is most commonly assessed visually by the dentist or a hygienist to assess gums for inflammation and bleeding, bacterial plaque, gum shrinkage and tooth mobility. It may further be monitored by trained professionals using a probe to determine the health of the soft tissue surrounding the teeth or radiography to monitor periodontal heath. Neither of these methods are appropriate to be applied by the patient themselves to determine gum health.

A number of computer implemented methods of dental plaque analysis have more recently been developed which make progress over the above conventional methods in providing more accurate and reliable measures of oral health. Such techniques generally utilise planimetric methods based on plaque percentage index (PPI) to analyse images of the teeth to determine plaque levels. This generally requires a specially designed positioner to ensure the image position is constant, allowing time series image data to be collected. Although such techniques have been shown to provide more reliable and accurate measurements of plaque levels than staining, the system is expensive and must have a skilled operator to obtain and analyse the images. Furthermore, such methods are primarily focussed on identifying dental plaque and therefore cannot determine all of the conditions described above. For example, malocclusion and gum conditions are generally not monitored by these systems. Another important issue is that such methods generally have significant data processing requirements. This may further prevent the methods being applied in a small handheld device such as a smartphone so as to be easily accessible to a patient. It may also inhibit the ability to provide an instant indication of areas of the mouth which require attention.

A further issue with known computer-implemented plaque identification methods is that they generally require an expensive camera and a trained operator to capture appropriate images for processing. One of the reasons for the requirement for expensive equipment and a trained operator is that the images must be collected with precise control over image parameters such as lighting, focal lengths, camera perspective, rotation of image and relative position of captured image area. Many systems also require plaque to be highlighted manually within the image processing software before a calculation of plaque level is made.

There accordingly exists a need for a system and method for monitoring both tooth and gum conditions, beyond solely the identification of plaque, which can be implemented by the patient themselves without requiring medical expertise or training. Furthermore the method must be able to measure a range of both tooth and gum health indicators whilst being non-time-consuming, inexpensive and straightforward to implement by the patient. There further exists a need for an efficient digital processing method which reduces the processing requirement to provide a quick, and reliable method which may be applied to images collected by an untrained user on a low cost imaging device. The method should also allow for instant feedback to be provided to the user, in particular to identify any regions of the mouth which require attention.

### SUMMARY OF THE INVENTION

The present invention seeks to provide a system and method for digitally processing an image of the teeth and gums to provide a reliable measurement of tooth and gum health. The invention seeks to provide a straightforward method which can be implemented by an untrained user to provide a quick and accurate estimate of tooth and gum health and also determine a change over time. The invention further seeks to provide a system which allows for an image to be captured from a reproducible position relative to the mouth with control over the various imaging parameters. The invention seeks to utilise a method which has a reduced data processing requirement in comparison to prior art techniques such that it can be implemented on a low cost imaging device, such as a smartphone, to give instant feedback to a user regarding their oral health. It is a further aim to provide the increases in processing efficiency whilst maintaining the required accuracy and flexibility such that a range of tooth and gum conditions can be identified, rather than focussing solely on the identification of dental plaque as common in the prior art.

According to a first aspect of the invention, there is provided a system for processing an image of the mouth, the system comprising: an image capture device; a mouthpiece attachment comprising a hollow tubular body with opposing first and second open ends, wherein the first open end is configured to be held in the mouth of a user and the second open end is configured to attach to the image capture device such that, in use, the image capture device images the user's mouth through the tubular body from a fixed position relative to the user's mouth; a display; and a processing unit; wherein the processing unit is configured to: receive mouth image data from the image capture device; identify an area of analysis within the image data corresponding to the teeth and gums; extract a set of image property features from the area of analysis of the image data; pass the image property features through a condition classifier, the condition classifier configured to compare the values of the extracted image property features against pre-set parameters to identify sub-areas of the area of analysis which are indicative of oral health conditions; and send result data corresponding to the identified sub-areas to the display.

The present invention therefore provides a system in which the imaging device is mounted in a fixed and reproducible orientation relative to the mouth by the use of a mouthpiece, such that a user has the required level of control over lighting, focal lengths, camera perspective, rotation of the image and the relative position of the captured area. This allows the images to be taken under reproducible conditions such that the reliability of the result data is increased and trends over time can be analysed. It further allows for a user to take the images themselves rather than requiring a skilled operator.

By using a tubular mouthpiece which is held in the mouth of a user, as defined, the user may reliably and reproducibly capture images of their mouth, without the aid of a skilled operator. Furthermore, the arrangement of the mouthpiece is such that the images are taken under reproducible conditions. For example, the lighting through the mouthpiece by a flash is constant and the image capture device is held in a constant position relative to the mouth, avoiding the need for a trained operator to ensure their imaging conditions are reproduced for processing.

By extracting a set of image property features which are passed through a classifying algorithm, a range of different oral health conditions may be identified rather than just plaque. A classifying algorithm can sort data into a plurality of classes where each class is defined by a specific combination of different image property feature values such that multiple conditions may be identified for both teeth and gums. A high reliability of identification can be achieved by careful setting of the pre-set parameters, which may be optimised via training the classifier through machine learning. The process of the current invention is also fully automated such that no manual input in the analysis of the images is required. The user is furthermore provided the desired instant visual indication of the presence of oral health indicators such as plaque and gum inflammation. The method can be extended to compare generated images collected over a period of time in order to give an indication of changes in the patient's oral hygiene. The simplicity and processing efficiency of the method means that it can be employed on a personal device such as a smartphone making it cost effective and accessible for a patient to implement themselves.

### BRIEF DESCRIPTION OF THE DRAWINGS

Embodiments of the invention will now be described, by way of example only, with reference to the accompanying drawings, in which:

FIG. 1 is a schematic diagram illustrating a system according to the present invention;

FIG. 2 is an illustration of a mouthpiece according to the present invention;

FIG. 3 is a flow diagram of a method configured to be executed by a processing unit of a system according to the present invention;

FIG. 4 is a flow diagram of a further method configured to be executed by a processing unit of a system according to the present invention; and

FIGS. 5A to 5E illustrate the display of result data with a system according to the present invention.

### DETAILED DESCRIPTION

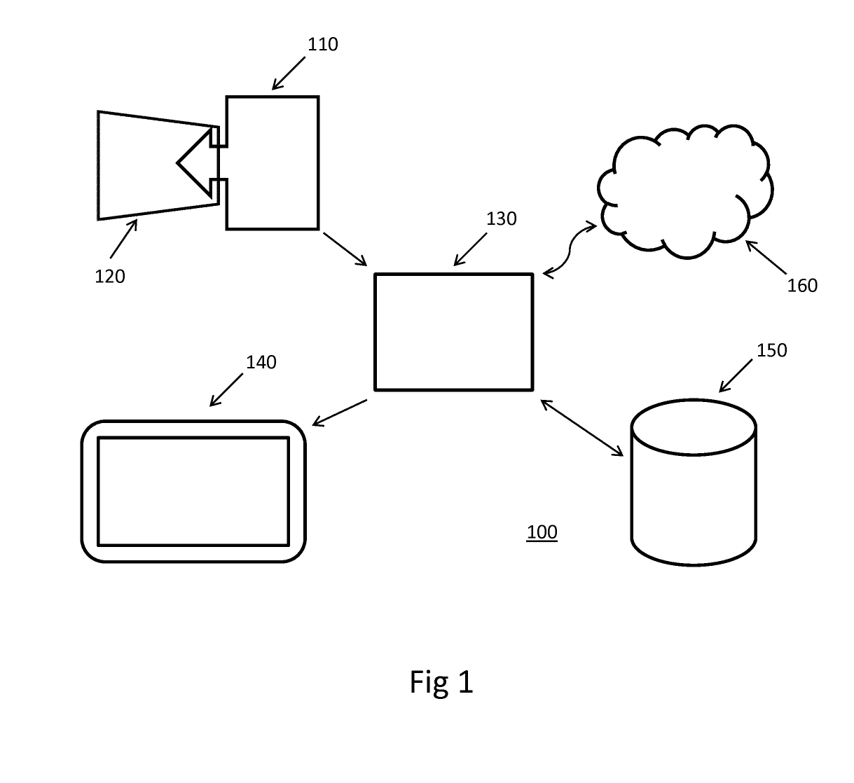

FIG. 1 is a schematic illustration of a system 100 according to the present invention. The system comprises an image capture device 110 configured to be attachable to a mouthpiece attachment 120, such that, in use, the mouthpiece 120 may be held in the user's mouth, mounting the image capture device 110 in a fixed position relative to the user's mouth. The system further comprises a processing unit 130 configured to receive mouth image data from the image capture device; identify an area of analysis within the image data corresponding to the teeth and gums; extract a set of image property features from the area of analysis of the image data; pass the image property features through a condition classifier, the condition classifier configured to compare the values of the extracted image property features against pre-set parameters to identify sub-areas of the area of analysis which are indicative of oral health conditions; and send result data corresponding to the identified sub-areas to the display where it may be displayed to a user for an instant indication of tooth and gum health. The system 100 may preferably include a memory unit 150 and the processing unit may further be configured to connect to a network such as the internet 160 to provide further functionality, as will be described.

The various components of the system may be separate components connected via direct or wireless connections. Alternatively they may be provided as an integrated handheld unit such as a smartphone.

The oral health conditions which may be identified with the system according to the present invention include one or more of: plaque levels (both new plaque or “biofilm” or old plaque known tartar), gingivitis, tooth decay (caries), periodontal disease, receding gums, teeth alignment, enamel wear and tooth discolouration.

These conditions may be associated with changes in texture, morphology and colour of the gums and teeth which may be detected as changes in the properties of mouth image data with the system according to the present invention.

Image Capture Device

The image capture device 110 is configured to acquire digital image data and send the image data to the processing unit. The image capture device 110 can be a specialist component provided for use with the system or can be a conventional image capture device such as a conventional digital camera, webcam, video camera or smart phone camera. The image data captured can be a single static digital image, an image sequence or a video. The image data is then sent to the processing unit. The data may be sent over a direct physical connection or via wireless communication such as Wi-Fi or Bluetooth. As will be described, in a preferred example, the system is provided via a smartphone using the smartphone camera, processing capability and display integral to the smartphone device.

A difficulty in capturing images for processing—particularly when these are captured by the user themselves—is the fact the images are taken under non-controlled conditions. Processing of the digital images can be hampered by variations in the image data, for example those caused by lighting conditions, focal lengths, camera perspective, rotation of the image, the relative position of the captured image area, camera resolution and blurring. Non-uniformity in the conditions under which the images are captured can result in a decreased reliability in successfully identifying oral health conditions since the various image property features used to determine their presence will vary greatly.

For these reasons a mouthpiece attachment 120 is used to mount the camera in a fixed position relative to the teeth and gums such that many of the above described image parameters remain constant between images. The mouthpiece therefore provides consistency in imaging conditions without requiring complex equipment or the aid of a skilled operator.

The system according to the present invention may also incorporate different types of image capture devices, either in addition or in place of a conventional digital camera. For example a 3D camera or depth sensing camera can allow for further morphology analysis to be implemented and improve detection of areas indicating oral health conditions. Furthermore an infrared camera can be used to enhance gum analysis.

Mouthpiece

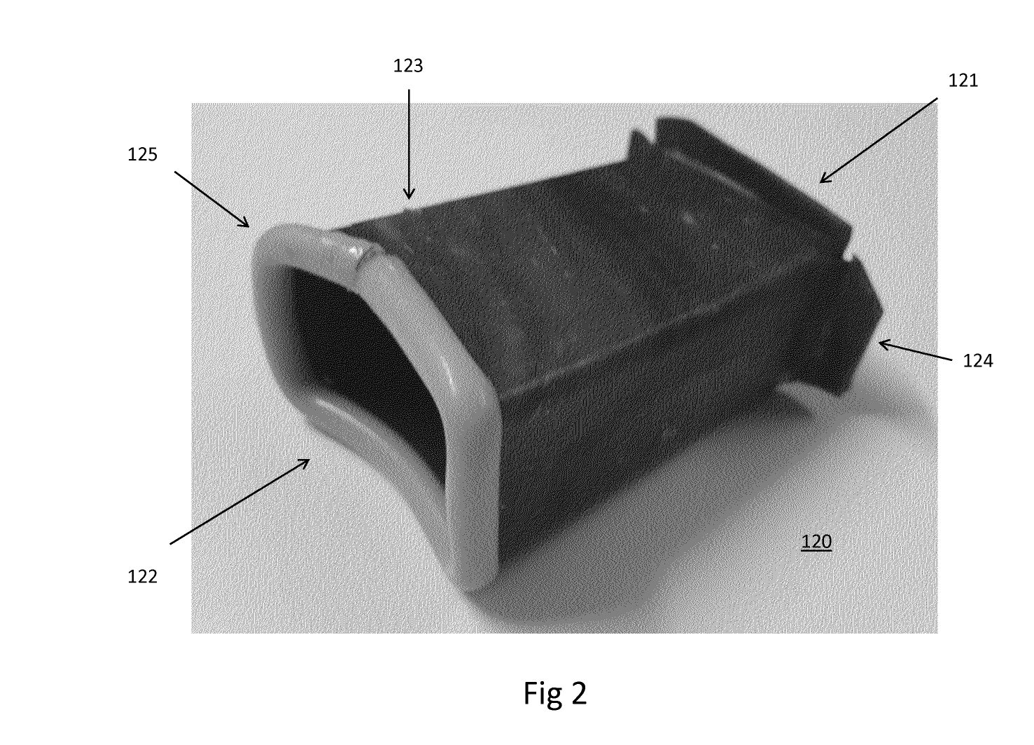

An exemplary mouthpiece 120 according to the present invention is illustrated in FIG. 2. The mouthpiece 120 may have a hollow, tubular body 123 with a first open end 121 configured to attach to the image capture device 110 and a second open end 122 configured to be received by the user's mouth. The mouthpiece 120 may further comprise an attachment means 121 adjacent to the first opening 121 of the mouthpiece 120, configured to attach to the image capture device 110 by any suitable means. For example it may slide on to the image capture device, clip on to the image capture device or be screwed into place. The attachment mechanism must be such that the lens of the image capture device 110 is held in a constant and reproducible position relative to the first opening 121 each time it is attached to the mouthpiece 120.

The mouthpiece may further comprise a rim 125 positioned around the perimeter of the second opening 122 of the mouthpiece. The rim 125 may comprise a tubular lip which frames the first opening and extends radially beyond the body of the mouthpiece. The rim 125 may take any other shape which preferably aids in allowing the user to hold the mouthpiece in a constant position with their lips positioned around the rim and may also be easy identifiable during the image processing steps to select the area of interest.

The rim is configured to be received by the user's mouth, such that the user's lips are placed around the rim 125 which rests against the user's gums. The size of the second opening is such that, when held with the user's lips around the rim, the opening frames the user's front teeth and a portion of the gums surrounding the front teeth. The mouth-interfacing plane of the second opening 122 may have a concave curvature such that the rim 125 follows the curvature of the gums when held in the user's mouth. The second opening 122 may have a curved rectangular shape with a concave curvature such that it conforms to the curvature of a user's gums. However, the second opening may take any other appropriate shape which conforms to the shape of the user's mouth to define an opening through which the teeth and gums may be imaged.

The rim 125 may act as a frame for the captured image, which may be utilised in the image processing steps executed by the processing unit, as will be described.

The arrangement of the rim 125 around the second opening 122 is such that, when the user's lips are placed around the rim 125, the attached image capture device 110 is held in a constant position relative to the teeth and gums. The image area is therefore constant between images. Similarly the distance of the image capture device 110 from the teeth and gums is constant providing a consistent focal length and rotation between images. The first, camera-interfacing opening 121 of the mouthpiece attachment 120 may be appropriately sized such that the camera flash also lies within the opening in addition to the aperture of the camera 110. In this way the lighting of the oral image may also be kept constant between different images, since the mouthpiece 120 excludes any ambient light from the surroundings meaning the lighting of the image is solely provided by the constant output of the flash. In other words, the second opening of the mouthpiece may be shaped so as to form a seal against the user and the first opening may form a seal around the camera and flash. In this way, no ambient light may reach the imaged area, other than that from the camera flash such that the lighting conditions remain constant.

In some examples of the mouthpiece of the current invention the rim 125 comprises a rubber border running around the second opening 122.

Image Processing Steps

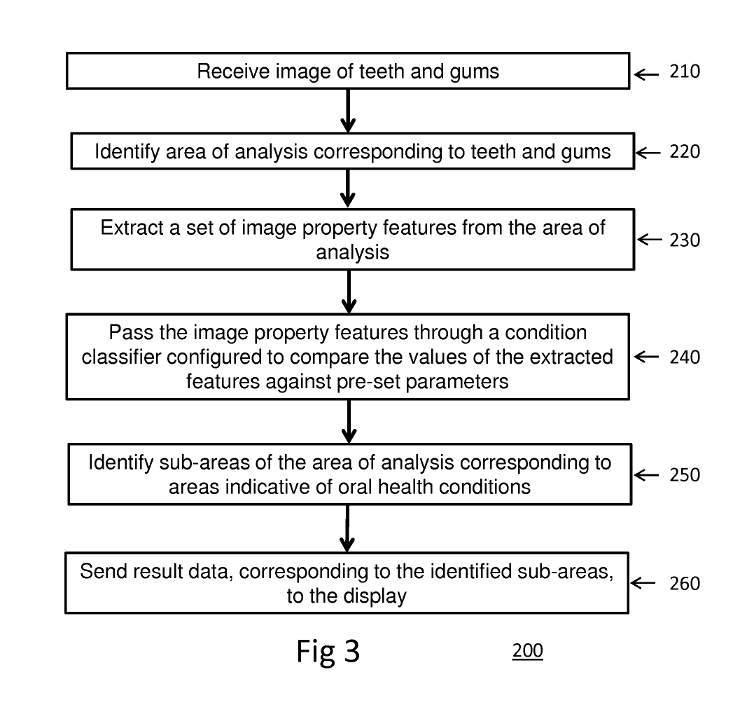

Once image data has been captured by the image capture device 110, the image data is sent to the processing unit 130 for processing. The processing unit 130 is configured to implement several image processing steps to determine areas of the image data which likely correspond to indicators of oral health conditions via analysis of the properties of the image data. The steps of a possible image processing method, executed by the processing unit of the current invention, are illustrated in the flow diagram of FIG. 3.

Before describing the various method steps in detail, the terminology used will first be defined.

An image property feature or feature of the image data is a measurement of a characteristic property of the image or part of the image. Examples of such properties are provided below.

A feature vector is an n-dimensional vector of numerical feature values which represents the image data or a portion of the image data.

A classifier is an algorithm configured to analyse the numerical values taken by various image property features within a vector and accordingly organise the corresponding parts of the image data into various categories. A classification algorithm consists of two phases of the process, training and testing. Initially in the training phase, characteristic image property features of the image or part of the image are isolated and based on these, a training class is created. Once this is established, the next step is the testing phase where these feature-space partitions are used to classify image features and group the data into the training classes.

A class is a unique description of a classification category. Classes are defined according to various parameters, where the parameters are values, ranges or relationships taken by one or more image property features or combinations of image property features. Such that a classifier analyses the set of image property features extracted from an image part, and compares them to the parameters which define each class. If the analysed image property features fall within the parameters which define a certain class, the classifier will sort the image portion into that class. Classes used in the context of the current invention, include teeth and gum classes for determining the parts of the image data which relate to the teeth and gums and various oral health condition classes which relate to specified oral health conditions.

A superpixel or cluster of pixels or pixel cluster is a group of neighbouring pixels such that an image may be segmented into clusters of pixels before analysis by a classifier.

An area of analysis refers to the part of the mouth image data being analysed, namely the parts corresponding to the teeth and/or gums.

A wide range of image property features have been studied in order to determine which image property features may be used to most reliably characterise image areas as belonging to the teeth or gum classes or range of oral health condition classes. One important type of image property features used is colour/texture descriptors these include:
statistical parameters on each color layer in different color spaces (e.g RGB, HSV), such as standard deviation and variance
Entropy
Measurements based on Gray Level Co-occurrence matrix such as energy, homogeneity, contrast and correlation
Haralick features
Linear Binary pattern

When extracting features these may correspond to the values of properties of single pixels, groups of pixels or the relationships between the values of neighbouring pixels. For example, Haralick texture features may be calculated for each pixel by comparing the relative values of neighbouring pixels. Similarly linear binary pattern calculations take into account the neighbouring pixel values.

Returning to the method flow diagram of FIG. 3, in the first step 210 the processing unit firstly receives the data sent, via direct connection or via wireless communication, from the image capture device 110.

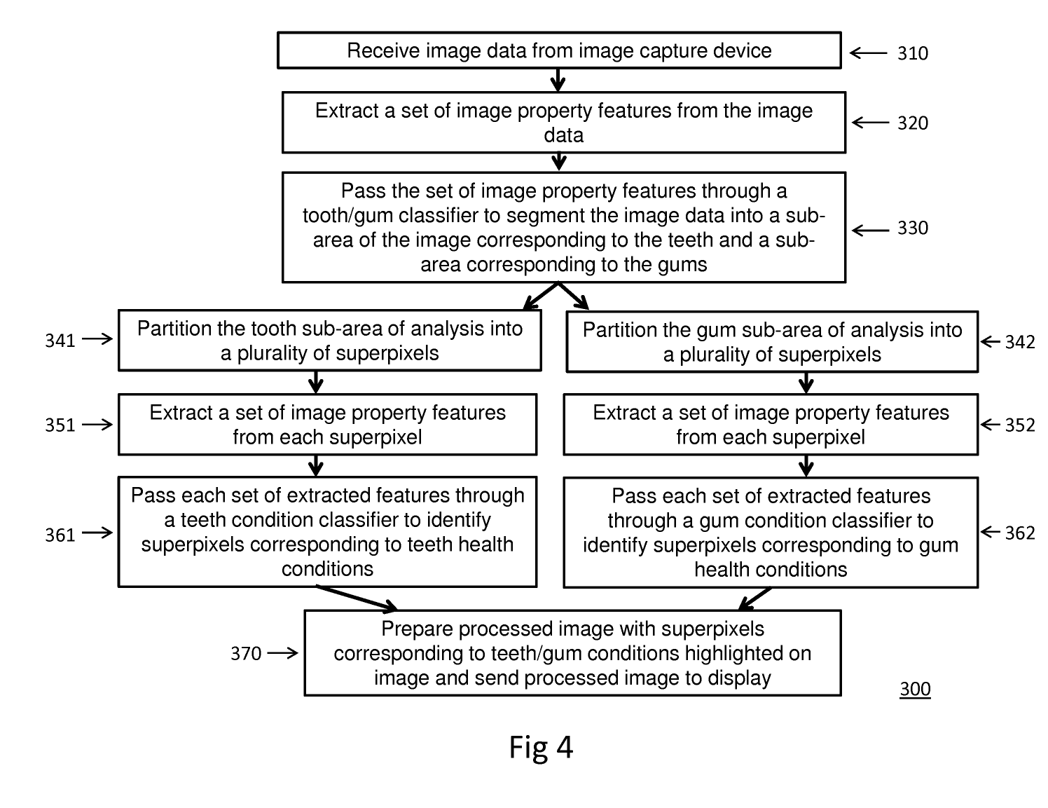

The processing unit then identifies the area of analysis within the image data in the second step 220. The area of analysis refers to the portion of the image data corresponding to the teeth and/or gums. The processing unit 130 may be configured to identify the area of analysis in several different ways. For example, the processing unit may simply detect the rim 125 of the mouthpiece 120 within the image data and simply determine that all image data within the frame of the rim 125 is to be used as the area of analysis. Alternatively more complex methods may be used. In a preferred example, image property features are extracted from the image data, placed in a vector and passed through a teeth/gum classifier where the teeth/gum classifier is configured to analyse the feature values to sort the image data into a class corresponding to the teeth, a class corresponding to the gums and a class corresponding to non-teeth/gums. More details of this method will be described with reference to FIG. 4.

After the portion of the image data corresponding to the teeth and/or gums has been identified as the area of analysis, the processing unit then extracts a set of n image property features from the area of analysis in step 230 and places these in a vector which represents the image data or a portion of the image data. Examples of the image property features may be those described above, which have been found to most reliably characterise the targeted oral health conditions. The feature vector contains the numerical values of each of these features for a data point of the image data in the area of analysis. Features may be extracted from each pixel, such that a feature vector is formed for each pixel or a feature vector may be formed from larger regions of the image data, as will be described with reference to FIG. 4. The relationships between the feature values of neighbouring data points may also be extracted into the feature vector and used to classify the data points.

In step 240 the feature vector is passed through the condition classifier which compares the values of the features against pre-set parameters to determine whether the corresponding areas of the image are indicative of oral health conditions.

The condition classifier is an algorithm which may be configured to sort the image data into one or more classes, where the classes are associated with certain oral health conditions and are defined by the pre-set parameters. The pre-set parameters may be prescribed ranges of numerical values that certain specified features must take for the corresponding image data to be sorted into that class. For example, the condition classifier may only place a portion of image data into the dental plaque class if the extracted standard deviation of a certain colour layer is within a pre-set range and the homogeneity and contrast of measurement on the Gray Level Co-occurrence matrix are also with certain ranges or below certain thresholds.

The extracted features may also be compared to the corresponding features of neighbouring data points such that the relationship between the features of different data points may also be used to classify the data.

In order to initially set the pre-set parameters which define the various classes of the classifier, the classifier may be trained by passing through extracted features to determine those image property features which most reliably characterise the required oral health conditions. Image data of known conditions may be passed through to determine which features and corresponding value ranges most reliably define the conditions. Furthermore, the classifier may self-optimise via machine learning over time. In particular, as more user data is passed through to optimise the parameters to more accurately and reliably detect the oral health conditions. Furthermore, the classifier may be optimised for a specific user, whereby the parameters are refined over time to more reliably identify oral health conditions for a specific user.

The condition classifier may be further configured to account for the varying reliability of using different features to identify different conditions. In particular, different weights may be assigned to differing feature parameters in the class definitions. Such that, for example, when classifying dental plaque, a greater weight may be given to whether the standard deviation of a certain colour layer lies within the predetermined parameters than the shape of an identified colour area, reflecting a greater reliability of the former feature in correctly identifying the presence of this condition.

In step 250, the sub-areas of the analysis area corresponding to areas indicative of oral health conditions are identified from the portions of the image data sorted into the various classes. For example all of the portions of image data sorted into the dental plaque class form an identified dental plaque sub-area.

In step 260, this data corresponding to the identified sub-areas, is sent to the display. The results data may be presented on the display in several different ways. In a preferred example, the identified sub-areas corresponding to each oral health condition are assigned a different colour and are overlaid on or combined with the original image data to be displayed to the user, thereby giving a near-instant visual indication of tooth and gum health, without requiring any complex equipment or expert operation. Since the image capture device, display and processing unit can be provided by a smartphone, to which the mouthpiece may be attached, the system according to the present invention provides an inexpensive system which provides a convenient method to allow the general population to instantly check the health of their teeth and gums and be able to track this overtime.

Various calculations may also be performed on the result data to provide quantitative measures of oral health conditions and sent to the display. For example the plaque percentage index may be calculated and displayed to the user, or a teeth or gum health score based on the extent of the conditions identified.

FIG. 4 illustrates a further, preferred method which the processing unit of the system according to the present invention may be configured to provide. The flow diagram of FIG. 4 illustrates further details of how certain processing steps may be achieved—in particular, how the areas of analysis may be identified and how the areas of analysis may be segmented into data points before classification. Although these additional details are both introduced here for means of illustration as part of the method diagram in FIG. 4, these additional step details are not interrelated and may be employed independently of each other.

In step 310 image data of the mouth is received from the image capture device.

A set of image property features is then extracted from the image data in step 320 and the n image property feature values are placed in a vector. As described above, the data points for which a vector is created may correspond to each pixel or larger regions of the image data.

In step 330, the features vectors are run through a tooth/gum classifier algorithm, the classifier configured to sort the image data into a tooth class (having parameters defined to correspond to tooth image data) and a gum class (having parameters defined to correspond to gum image data).

The configuration of the tooth/gum classifier and the image property features used may take various different forms to provide the key technical result of segmenting the image data into data corresponding to the teeth and data corresponding to the gums. Segmentation methods used by the tooth/gum classifier include:
Thresholding, where the classifier sorts the image data based on threshold values of certain features, specific to the tooth or gum areas of analysis. For example, the classifier may sort a data point into the tooth class if the standard deviation in a certain colour layer is below a certain threshold value;
Histogram methods, where histograms are computed in different colour maps and statistical features are extracted that can be used to identify boundaries between teeth and gums;
Edge detection, where the classifier compares the differences between neighbouring data points and identifies the presence of a sharp intensity change as indication of the boundary between teeth and gums; and
Watershed transformation, where the classifier identifies pixels with the highest gradient magnitude intensities, which represent region boundaries between areas of analysis.

Once the image data points have been classified into one of the classes—either teeth, gums or non-teeth/gums—these data points are combined to form a tooth sub-area of analysis and a gum sub-area of analysis. These may be thought of as a sub-image solely of the teeth and a sub-image solely of the gums, which may each be analysed individually by a specialised classifier. The method diagram of FIG. 4 therefore splits into two analysis paths, the first corresponding to the teeth image data and the second corresponding to the gum image data. Since the teeth image data and the gum image data may be analysed independently, this allows the analysis for each data type to be specialised to identify conditions solely associated with either the teeth or gums. For example it allows quantitative calculations to be applied to the teeth only or the gums only, one example of which is the calculation of PPI for the teeth.

In steps 341 and 342 the image data corresponding to the teeth and gums respectively is portioned into a plurality of clusters of pixels or superpixels. As described above, each pixel of the image data may provide a data point from which a feature vector is extracted, alternatively, as in the example if FIG. 4, the image data may be partitioned into larger sub-areas, or superpixels, corresponding to a plurality of pixels, from which a feature vector is extracted. Partitioning may be performed by clustering methods, in which the image data is split into clusters of image pixels. The clusters may be a constant number of pixels or they may vary, for example, where the image data is split into clusters having similar feature values. The segmentation methods described above may therefore also possibly be used to the portioning of the data into clusters of pixels.

The partitioning into clusters of pixels or “superpixels” allows for certain analyses to be carried out not only on single data points or pixels but for the relationships between the features of pixels within the superpixel to be extracted and analysed.

After the tooth area of analysis and the gum area of analysis have both been portioned into a plurality of superpixels, in steps 351 and 352 a set of image property features is extracted from each superpixel and placed into a feature vector.

In steps 231 and 362 the extracted feature vectors for each superpixel are placed through a teeth condition classifier and gum condition classifier respectively. Both the teeth and gum condition classifiers are configured in a similar principle to the condition classifier described with reference to FIG. 3. Each is configured to compare the values within the feature vectors to the class parameters which define the portion of the feature-space which corresponds to each specified oral health condition. Here the classifiers may be specialised to identify teeth conditions and gum conditions respectively. For example the tooth condition classifier may only analyse features relevant to the defined tooth condition classes and the gum condition classifier may only analyse features relevant to the defined gum condition classes, thereby providing an increased reliability in correctly identifying specific conditions. By parallel processing the image data in this way, a fast and efficient method is provided with a reduced processing capability requirement.

In addition to extracting features from each set of pixels, the morphology/shape of each area of analysis is analysed whereby specific morphological features may be identified to locate indication of oral health condition. For example, the shape of the gum area of analysis may be analysed to identify gum recession or the shape of the teeth area of analysed may be analysed to identify changes in tooth alignment which may be indicative of certain oral health conditions. The shape analysis may also be applied to superpixels or sub areas of the areas of analysis with the results extracted as features used to classify the corresponding portions of image data. Various techniques may be used to analyse the shape of the tooth area of analyses. The shape analysis techniques may include one or more of:
the calculation of Hough transforms, a technique that isolates features of a particular shape;
parabolas analysis, a technique involving the calculating of the gradient of parabolas and curves, which may be used as identifiers of specific morphological feature indicative of certain conditions; and
shading analysis, a technique which uses light and how light responds to the shape of regions in an image as an identifier of certain conditions.

The system can further analyse the changes in the shape of the areas of analysis over time as result data can be saved in a local 150 or external 160 memory, as will be described below. The system can enable the use of shape matching to analyse changes or transformation that occurs to individual users overtime and provide further indicators of teeth and gum health.

In step 370, the clusters of pixels (superpixels) associated with each of the specified oral health conditions are highlighted, for example by colouring superpixels belong to each class with a different colour. The coloured superpixels, corresponding to the oral health conditions, are then combined or overlaid on the original mouth image and sent to the display to provide the user with an instant indication of their oral health.

In addition to purely visual qualitative result data, the processing unit 130 may also calculate a number of quantitative measures of oral health. For example, the plaque percentage index may be calculated and included in the result data or scores relating to the relative presence and severity of other selected conditions.

A number of additional features may be included in the system according to the present invention. For example the system may include at least one of a local memory storage unit 150 or connectivity to connect to a network such as the internet 160. The generated result data may then be saved to the local storage 150 or uploaded to a user's profile on the internet. The inclusion of a memory allows for a personalised library and unique data set to be built for each user. By accessing saved user data from the memory 150, 160 the trends in the user data over time may be tracked and changes that are uncharacteristic may be identified and located. Example of uncharacteristic changes may include the gum line receding or teeth alignment changes.

Each time a user analyses an image of their mouth the result data may be saved into the local or network storage, thereby compiling a library of user data. This allows the user to analyse changes in their oral health, such as plaque levels, gingivitis or teeth whiteness over time. Since the mouthpiece provides consistent and reproducible imaging conditions, accurate comparisons between result data may be made, since the imaging conditions are constant such that changes in the data are solely or substantially due to differing oral conditions.

The assembly of a user data library also allows the classifiers to be configured according to a specific user. For example the feature values according to gingivitis might vary from user to user, or change over time, therefore the parameters used by the classifier to define the various classes may be altered to reflect the values which most accurately categorise the specified conditions for that user at that time.

The result data collected may also be sent to a central database, via the internet connection 160, such that large scale trends in oral health data may be collected. For example a population-wide mode of features may be collected giving an understanding of what is uncharacteristic on a population-wide level. The data may further be used to determine which oral health conditions certain populations are susceptible to, aiding a demographic understanding of oral health conditions.

The connection to an external network 160 may also allow the user's health data to be sent to a dentist or other health care professional for analysis or flag if certain thresholds are exceeded, indicating medical attention is required. The system can furthermore support or automate check-ups carried out by an in-clinic healthcare professional.

The system may also include a user interface such that the user can navigate a menu to select various options regarding the data processing and result display. For example the user may select specific oral health conditions, for example dental plaque level or gingivitis, that they would like to analyse the image data to identify. They may search for all health conditions and then display the results independently on the displayed image. They may further make selections to look at the changes in the various condition results over time.

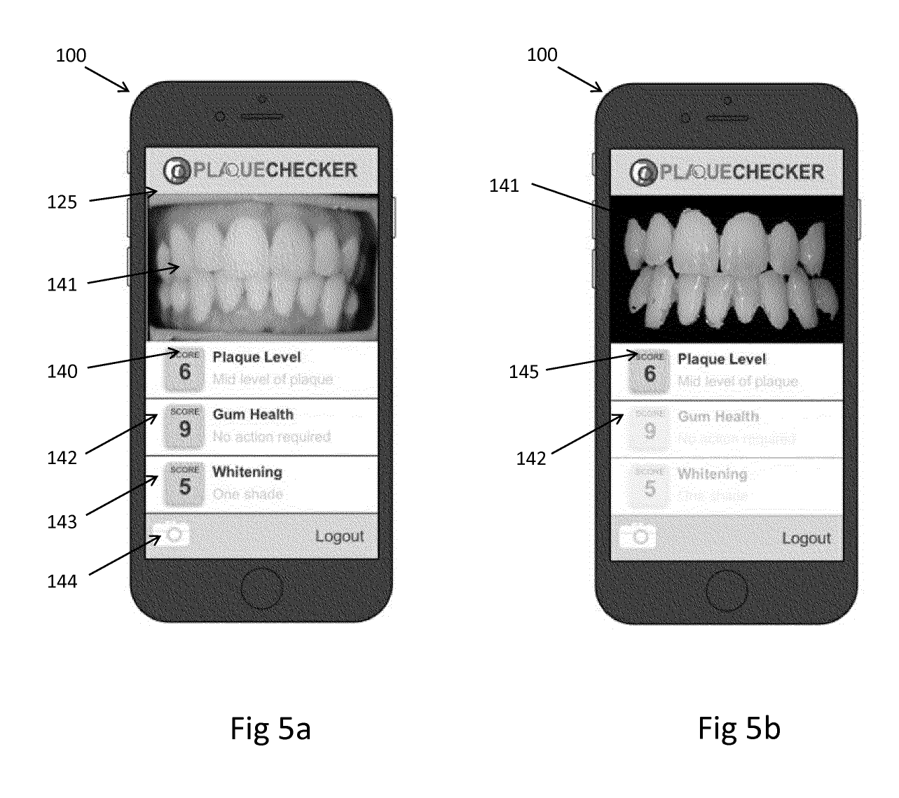

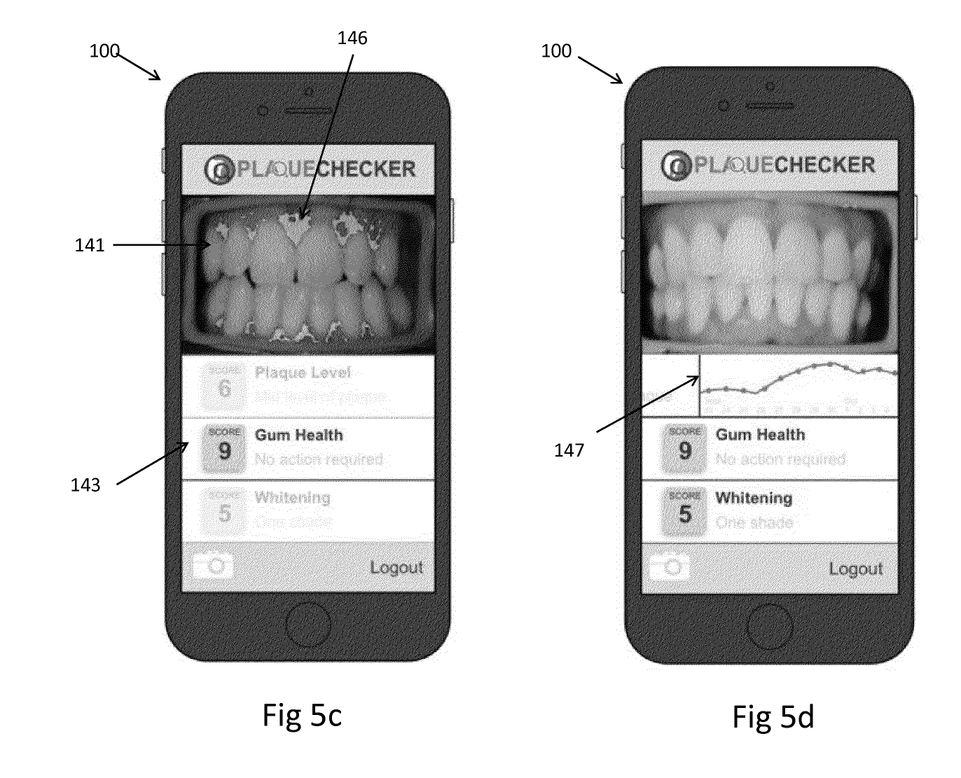

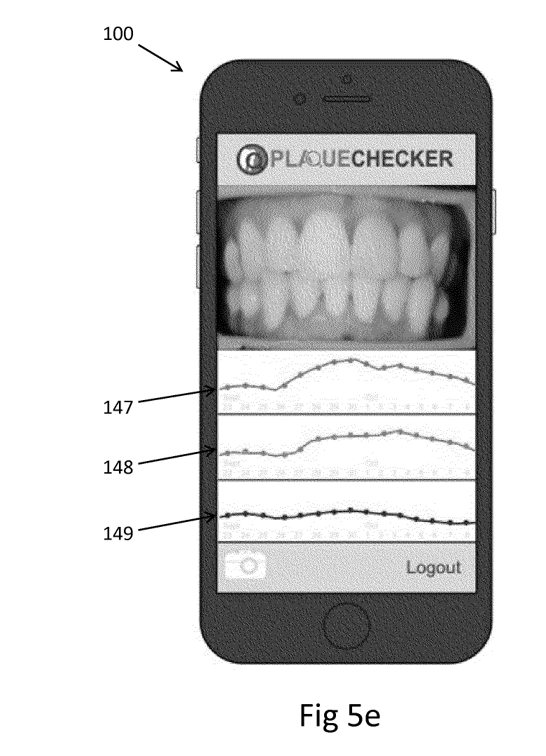

FIGS. 5A to 5E show a system according to the present invention, illustrating the display of user result data.

As described above and illustrated in FIG. 5, in one example of the present invention, the various components are integrated into a singular device such as a smartphone, where the functionality carried out by the processing unit 130 is provided via an application (or “app”) running on the smartphone, with the image data collected by the smartphone camera and the result data displayed on the screen 140 of the smartphone.

FIG. 5A shown an image captured by the image capture device displayed on the smartphone display 140. As clear from a comparison of FIGS. 5A to 5E, the mouthpiece attachment 120 ensures that the image capture device (in this case the smartphone camera) is aligned in a constant, reproducible position relative to the teeth and gums. The rim 125 of the mouthpiece 120 is visible in the display image, which frames the target area and may be used as a reference point in the analysis.

In this example the captured image 141 is displayed in a portion of the display to the user. Also a number of quantitative measures are displayed, in this case, a plaque level score 142 a gum health score 143 and a whitening score 144. These metrics may be calculated from the result data, for example from the level and intensity of the related oral health condition detected.

The user may use the user interface (in this example the touch screen of the smartphone) to select various options in terms of the result data to be displayed. For example, the user may select plaque level, to display the detected plaque superimposed on the mouth image 141, as shown in FIG. 5B. Here, the portions of the image data determined as areas of plaque by the classifier are highlighted 145 on the mouth image 141 in a different colour. The plaque level score 142 is simultaneously displayed and a brief summary of the plaque level is provided—in this case “mid-level of plaque”. The plaque score may be related to the plaque percentage index.

The user may alternatively select “gum health”, as shown in FIG. 5C, such that areas 146 of the gums possible indicating the presence of gum conditions such as gingivitis are displayed as colour sections on the mouth image 141. A quantitative score 143 relating to the level of detected gum conditions may also be simultaneously displayed.

In addition to the results of the current analysis, historical or recent trend data may be displayed. For example, the user may select or swipe the present plaque score portion of the display 142 to switch to a graph showing the user's plaque level score over the preceding week or months 147, as shown in FIG. 5D. In this case the system accesses the result data stored in the local memory 150 or cloud 160 to plot the result data graph.

As shown in FIG. 5E, trends in result data may also simultaneously be displayed for other oral health indicators, for example gum health 148 and whitening 149 scores.

With the system according to the present invention, an inexpensive and convenient means is provided for a user to check their oral health, without requiring complex kit or the help of a skilled operator or trained health professional. The system allows the imaging conditions to be accurately controlled such that digital processing methods may be applied to reliably detect indications of oral health conditions. This furthermore allows the process to be fully automated such that the possibility of user error and associated inaccuracy can be eliminated. Since identification is carried out by extracting a range of image property features and using these to classify image data into categories defined by pre-set parameters, a wide range of oral health conditions can be identified by appropriate control of the specific features used and the parameters used to define the classes. This image analysis process further allows the identification of oral conditions to be optimised over time and for an individual user, enhancing the reliability of detection. The configuration of the processing unit furthermore provides a fast and efficient data processing method which can be implemented by a user on a device such as a smartphone to provide instant feedback regarding their oral health.

1. A system for processing an image of the mouth, the system comprising:
an image capture device;
a mouthpiece attachment comprising a hollow tubular body with opposing first and second open ends, wherein the first open end is configured to be held in the mouth of a user and the second open end is configured to attach to the image capture device such that, in use, the image capture device images the user's mouth through the tubular body from a fixed position relative to the user's mouth;
a display; and
a processing unit; wherein the processing unit is configured to:
receive mouth image data from the image capture device;
identify an area of analysis within the image data corresponding to the teeth and gums;
extract a set of image property features from the area of analysis of the image data;
pass the image property features through a condition classifier, the condition classifier configured to compare the values of the extracted image property features against pre-set parameters to identify sub-areas of the area of analysis which are indicative of oral health conditions; and
send result data corresponding to the identified sub-areas to the display.

2. The system of claim 1, the image capture device further comprising:
a flash to illuminate the user's mouth during imaging, wherein, in use, the mouthpiece attachment forms a seal against the users mouth around the first opening and the mouthpiece attachment forms a seal around a lens and the flash of the image capture device around the second opening.

3. The system of claim 2 wherein, in use, the sealed openings prevent ambient light entering the mouthpiece such that the user's mouth is illuminated solely by the flash during imaging.

4. The system of claim 1 wherein the mouthpiece attachment further comprises a tubular lip which runs around the circumference of the first open end, the tubular lip configured to be held within the user's lips during use to form the seal.

5. The system of claim 4 wherein the lip is adapted such that it may be identified by the processing unit within the image data such that the lip defines the outer border of the image data used in the analysis.

6. The system of claim 1 wherein the first opening of the mouthpiece has a size and shape such that, when held in the mouth of the user, the imaging device images the buccal surfaces of the teeth and gums through the first opening.

7. The system of claim 6 wherein the first opening of the mouthpiece attachment has a curved rectangular shape, with a concave curvature such that it conforms to the curvature of a user's gums when held in the mouth.

8. The system of claim 1 wherein the mouthpiece attachment further comprises
attachment means, configured to attach to the image capture device at the second opening of the device.

9. The system of claim 8 wherein the attachment means comprises at least one of:
a slide attachment; and
a clip attachment.

10. The system of claim 1 wherein, before extracting image property features, the processor is further configured to:
partition the area of analysis into a plurality of clusters of pixels, each cluster of pixels comprising a group of pixels;
extract a set of image property features for each cluster of pixels;
pass each set of image property features through the condition classifier to identify the clusters of pixels indicative of oral health conditions.

11. The system of claim 10 wherein the condition classifier is configured to sort the clusters of pixels into a plurality of classes, each class corresponding to a specified oral health condition; wherein
each class is defined by one or more predetermined class definition parameters, the parameters corresponding to numerical values taken by one or more image property features, or the relationships between the values of image property features in neighbouring clusters of pixels, which characterise the image data as belonging to that class; such that the classifier places a cluster of pixels into a class if the specified combination of image property features take values within the predetermined parameters.

12. The system of claim 11 wherein the class definition parameters are pre-set by passing image data of known oral health conditions through the condition classifier to determine the image property features, values of those image property features and relationships between neighbouring clusters of pixels which most reliably characterise the corresponding oral health condition.

13. The system of claim 1, wherein upon receiving the mouth image data, the processing unit is further configured to:
segment the image data into two areas of analysis, a first area corresponding to the teeth and a second area corresponding to the gums;
extract a set of image property features from both the tooth area of analysis and the gum area of analysis;
pass each set of image property features through a corresponding condition classifier to identify sub-areas of the teeth area indicative of tooth health conditions and sub-areas of the gum area indicative of gum health conditions.

14. The system of claim 13, wherein, to segment the image data, the processing unit is further configured to:
extract a set of image property features from the image data;
pass the image property features through a tooth/gum classifier, the tooth/gum classifier configured to compare the values of the image property features against pre-set parameters to identify areas of the image corresponding to the teeth and areas of the image corresponding to the gums.

15. The system of claim 14 wherein the tooth/gum classifier is configured to sort the image data into at least two classes, a tooth class and a gum class; wherein
each class is defined by a one or more class definition parameters, the parameters corresponding to values taken by one or more image property features which characterise the image data as belonging to that class; such that the classifier places image data into a class if the specified combination of image property features take values within the predetermined parameters.

16. The system of claim 1 wherein the processing unit is further configured to:
generate a processed image corresponding to the original image data with the sub-areas of the image indicative of oral health conditions highlighted; and send the processed image to the display.

17. The system of claim 1 wherein the processing unit is further configured to:
calculate one or more quantitative measures of oral health; and
send the quantitative measures of oral health to the display as part of the result data.

18. The system of claim 13 wherein the processing unit is further configured to:
analyse the morphology of the teeth area of analysis and gum area of analysis;
compare the morphology against pre-set parameters to identify whether the measured shapes are indicative of oral health conditions; and
send result data corresponding to the shape analysis to the display.

19. The system of claim 1 wherein the image property features include one or more of:
standard deviation of each colour layer in different colour spaces;
variance of each colour layer in different colour spaces;
other statistical parameters of each colour layer in different colour spaces; entropy;
measurements based on Gray level co-occurrence matrix, including one or more of: energy homogeneity contrast and correlation; and
Haralick features.

20. (canceled)

21. The system of claim 1 wherein the processing unit is further configured to compare result data against result data saved in a memory to provide a measure of the change in oral health conditions over time.

22. (canceled)

23. (canceled)
